(19) United States
(12) Patent Application Publication
(10) Pub. No.: US 2022/0234979 A1
(43) Pub. Date: Jul. 28, 2022

(54) PROCESS FOR THE PURIFICATION OF EUGENOL AND NOVEL COMPOSITIONS COMPRISING EUGENOL

(71) Applicant: RHODIA OPERATIONS, Aubervilliers (FR)

(72) Inventor: Valery DAMBRIN, Irigny (FR)

(21) Appl. No.: 17/610,754

(22) PCT Filed: May 14, 2020
(86) PCT No.: PCT/EP2020/063521

§ 371 Date: Nov. 12, 2021

(30) Foreign Application Priority Data
May 17, 2019 (FR) FR1905219
Sep. 27, 2019 (EP) 19200216.0

(51) Int. Cl. C07C 41/42; C07C 41/46

(52) U.S. Cl. CPC C07C 41/42; C07C 41/46

(57) ABSTRACT

The present invention relates to a process for the purification of crude eugenol by distillation in the presence of at least one stabilizing compound. The process of the present invention may be operated industrially, exhibits improved properties, in particular in terms of process safety, and makes possible efficient separation of ortho-eugenol. The present invention also relates to compositions comprising eugenol.

The present application claims the priority of the patent application filed on May 17, 2019 in France under the number 1 905 219 and of the patent application filed in Europe on Sep. 27, 2019 under the Ser. No. 19/200,216.0, the content of which is incorporated in full by reference.

### TECHNICAL FIELD

The present invention relates to a process for the purification of eugenol and novel compositions comprising eugenol.

### PRIOR ART

Eugenol is a major aromatic compound of many essential oils, such as clove oil. Eugenol is frequently used in perfumery but it also exhibits antiseptic, antibacterial, analgesic and antioxidant properties. It may also be used as a synthetic intermediate, in particular in the preparation of vanillin.

In view of this broad field of exploitation, it is necessary to produce eugenol on an industrial scale and to have available optimized manufacturing processes.

Classically, eugenol is of natural origin, extracted from plants. The extraction may in particular be carried out by hydrodistillation, extraction assisted by microwave or extraction with a supercritical fluid. The document CN 104326885 describes a process for the extraction of eugenol from an oil in which an aqueous phase containing a sodium salt of eugenol is produced and distilled. Alternatively, eugenol may be obtained by a chemical process, such as described in the document FR 2 302 991, in which guaiacol is allylated in the presence of allyl chloride and of a catalyst. Eugenol may be purified by distillation in order to be separated from the byproducts of the reaction. The document CN 108383695 also describes a chemical process for the preparation of eugenol. This document describes a process for the purification of eugenol in which an antioxidant is added to the purified eugenol in order to prevent coloring of the eugenol. The document CN 105294409 describes an alternative synthesis process which uses a composite catalyst based on copper and on cobalt. The document GB 1 501 221 describes another chemical process for the preparation of eugenol from guaiacol and allyl chloride.

When these synthesis processes are used, several side reactions may occur resulting in the formation of ortho-eugenol or O-allylation compounds. As a result, the purification of the eugenol is complex, in particular the separation by distillation of eugenol and ortho-eugenol. This purification is made all the more complex as eugenol and/or ortho-eugenol exhibits undesirable properties, in particular may be explosive under the operating conditions of the distillation. The document CN 105294409 indicates a purification process in which eugenol is purified using n-octane and K₂CO₃, the mixture is filtered, then washing with water of the K₂CO₃ is carried out and finally the purified eugenol is distilled from the aqueous phase.

This process exhibits the disadvantage of generating effluents, and does not seem very appropriate in the context of an industrial exploitation. The present invention is targeted at the efficient and selective manufacture of eugenol, in particular at a process making possible the efficient separation of eugenol and ortho-eugenol, on the industrial scale, under good safety conditions.

### BRIEF DESCRIPTION OF THE INVENTION

A first subject matter of the present invention relates to a novel process for the purification of eugenol in which a crude eugenol is distilled in the presence of at least one stabilizing compound.

The present invention also relates to a process for the separation of eugenol and ortho-eugenol by distillation in the presence of at least one stabilizing compound.

Finally, the present invention relates to a composition comprising eugenol and between 0.1 and 10 000 ppm of at least one stabilizing compound or of at least one stabilizing compound and of at least one auxiliary compound, in which the stabilizing compound is chosen from the group consisting of phenol derivatives, phenothiazine derivatives, TEMPO derivatives, CB, para-benzoquinone, para-phenylenediamine, diethylhydroxylamine, manganese(II) acetate, sodium nitrite, sodium ascorbate, potassium ascorbate, ergosterol, cholecalciferol and glutathione and the auxiliary compound is chosen from the group consisting of vitamin E, butylated hydroxyanisole (BHA), dibutylated hydroxytoluene (BHT) and tert-butylhydroquinone (TBHQ).

### DESCRIPTION OF THE FIGURES

FIG. 1 is a graph of analysis by differential scanning calorimetry of a sample of eugenol according to example 1.

FIG. 2 is a graph of analysis by differential scanning calorimetry of a sample of eugenol according to example 2.

### DETAILED DESCRIPTION

In the context of the present invention, and unless otherwise indicated, the expression “of between . . . and . . . ” includes the limits. Unless otherwise indicated, the percentages and ppm are percentages and ppm by weight.

In the context of the present invention, and unless otherwise indicated, the term “ppm” means “parts per million”. This unit represents a fraction by weight: 1 ppm=1 mg/kg. In the context of the present invention and unless otherwise indicated, the term “eugenol” refers to para-eugenol or 4-allyl-2-methoxyphenol according to the formula (I):

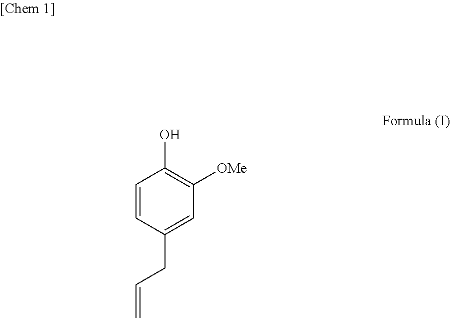

In the context of the present invention, ortho-eugenol refers to 2-allyl-6-methoxyphenol according to the formula (II):

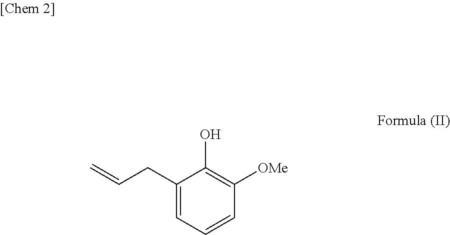

In the context of the present invention, the term “phenol derivative” represents a compound comprising a unit corresponding to the formula (III):

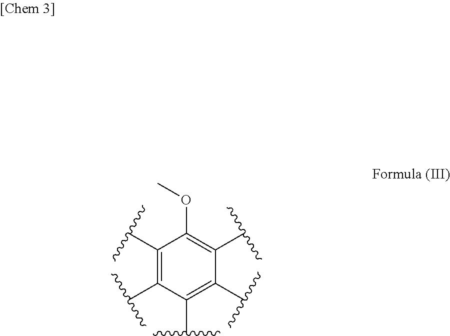 [SMILES: *c1c(*)c(*)c(OC)c(*)c1*]

According to a specific aspect, the term “phenol derivative” refers to a compound according to the formula (IV):

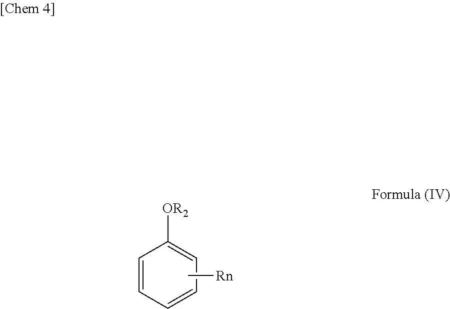

in which R represents a hydroxyl, a linear or branched alkyl, preferably comprising between 1 and 6 carbon atoms, alkenyl, an alkoxy group, preferably comprising between 1 and 6 carbon atoms, a nitro group or a nitroso group, R₂ represents a linear or branched alkyl, preferably comprising between 1 and 6 carbon atoms, and n is of between 0 and 5; preferably, n is equal to 0, 1, 2 or 3. Preferably, the R group is chosen from the group consisting of hydroxyl, methyl, ethyl, propyl, n-butyl, t-butyl, methoxy, ethoxy, propoxy, isopropoxy, nitro and nitroso. Preferably, the R₂ group is chosen from methyl, ethyl, propyl, n-butyl or t-butyl.

According to an alternative form, the term “phenol derivative” refers to a compound according to the formula (IV) above in which R₂ represents H and R is as defined above.

According to another specific aspect, a phenol derivative may be chosen from the compounds of the family of the tocopherols, in particular α-tocopherol, β-tocopherol, γ-tocopherol or δ-tocopherol.

In the context of the present invention, the term “phenothiazine derivative” refers to compounds comprising the unit according to the formula (V):

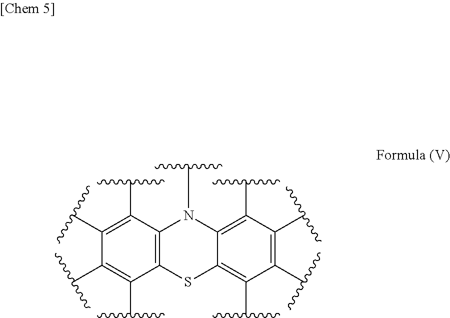 [SMILES: *c1c(*)c(*)c2c(c1*)Sc1c(*)c(*)c(*)c(*)c1N2*]

In the context of the present invention, the term “TEMPO derivative” refers to compounds comprising the TEMPO unit according to the formula (VI):

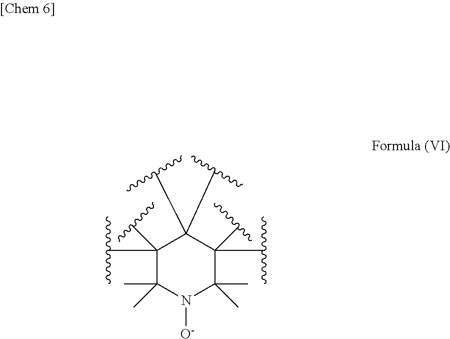 [SMILES: *C1(*)C(C)(C)N([O-])C(C)(C)C(*)(*)C1(*)*]

In particular, the term “TEMPO derivative” refers to (2,2,6,6-tetramethylpiperidin-1-yl)oxy (TEMPO), to (4-hydroxy-2,2,6,6-tetramethylpiperidin-1-yl)oxy (TEMPO—OH), to (4-oxo-2,2,6,6-tetramethyl-1-piperidin-1-yl)oxy (4-Oxo-TEMPO), to 4-amino-2,2,6,6-tetramethylpiperidine-1-oxyl(4-amino-TEMPO) or to bis(2,2,6,6-tetramethyl-4-piperidyl) sebacate (Bishydroxy TEMPO sebacate).

In the context of the present invention, the term “stabilizer” refers to a compound capable of maintaining the qualities of a product without affecting said product, such as the coloring, the chemical or thermal stability, in particular during the manufacture, the storage or the handling of the product.

In the context of the present invention, the term “crude eugenol” refers to a composition comprising essentially eugenol, in particular comprising at least 20% of eugenol, preferably at least 30% by weight of eugenol, more preferentially still at least 40% by weight and more preferentially still at least 50% by weight, with respect to the weight of the composition. Generally, the predominant compound of crude eugenol is para-eugenol. In particular, the crude eugenol according to the present invention may contain impurities, in particular related to the process for the synthesis of the eugenol, such as o-eugenol, or product of O-alkylation of guaiacol or eugenol; the impurities may also originate from the starting compounds used in the reaction. In general, the content by weight of each impurity present in the composition, with respect to the weight of the composition, is less than the content of eugenol in the composition.

In the context of the present invention, the term “purified eugenol” refers to a composition comprising essentially eugenol, in particular comprising at least 80% of eugenol, preferably at least 90% by weight of eugenol, more preferentially still at least 95% by weight and more preferentially still at least 99% by weight, with respect to the weight of the composition. In particular, the purified eugenol according to the present invention may contain impurities, in particular related to the process for the synthesis of the eugenol, such as o-eugenol, or product of O-alkylation of guaiacol or eugenol; the impurities may also originate from the starting compounds used in the reaction. The process according to the present invention makes it possible to purify crude eugenol, making it possible to obtain a purified eugenol.

The crude eugenol to be purified according to the present invention may be obtained by any synthesis process. The synthesis process may be carried out continuously or semi-continuously, in particular in a suitable reactor, such as a microreactor.

In a specific aspect of the present invention, the eugenol is obtained from guaiacol and allyl halide, preferentially allyl chloride, as described in the document FR 2 302 991. In particular, the alkylation reaction is carried out in the presence of an aqueous solution of an alkali metal or of an alkaline earth metal hydroxide, such as NaOH or KOH. In addition, the reaction may be carried out in the presence of a catalyst, in particular a copper-based catalyst, such as CuCl, CuCl₂.2H₂O, Cu(NO₃)₂ or Cu(OAc)₂.2H₂O. The reaction may also be carried out in the presence of a composite catalyst such as described in the patent application CN 105294409.

Generally, the amount of catalyst is greater than or equal to 0.01% by weight, preferentially greater than or equal to 0.02% by weight, more preferentially greater than or equal to 0.05% by weight and very preferentially greater than or equal to 0.1% by weight, with respect to the amount of guaiacol. Generally, the amount of catalyst is less than or equal to 10% by weight, preferentially less than or equal to 5% by weight, more preferentially less than or equal to 2% by weight and very preferentially greater than or equal to 1% by weight, with respect to to the amount of guaiacol. Generally, the temperature of the reaction is greater than or equal to 5° C., preferably greater than or equal to 10° C., more preferentially greater than or equal to 15° C. and very preferentially greater than or equal to 25° C. Generally, the reaction temperature is less than or equal to 95° C., preferably less than or equal to 80° C., more preferentially less than or equal to 65° C. and very preferentially less than or equal to 50°.

The reaction is generally carried out in the presence of an ammonium salt or of ammonia as described in the document FR 2 302 991. Ammonia may form a complex of copper-amine type with the catalyst.

In order to limit the generation of effluents having a high content of nitrogenous derivatives, it is possible to replace the ammonia and to carry out the reaction in the presence of glucose, glucosamide, citric acid or tartaric acid, 2-hydroxypyridine or nicotinamide.

Generally, initially, a guaiacol salt is formed in aqueous solution. The catalyst is added to the reaction mixture followed by the addition of an aqueous ammonia solution; finally, the allyl halide is added to the reaction mixture.

A first subject matter of the present invention relates to a process for the purification of a crude eugenol by distillation in the presence of at least one stabilizing compound.

According to another aspect, the present invention relates to a process for the purification of a crude eugenol by distillation in the presence of at least one stabilizing compound and/or of an auxiliary compound.

An aim of the present invention is in particular:
to provide a process for the manufacture and/or purification of eugenol which is improved in that it makes possible good productivity,
to provide a process for the manufacture and/or purification of eugenol which is improved in that it makes it possible in particular to obtain eugenol separated from the ortho-eugenol isomer,
to provide a manufacturing and/or purification process which is advantageous due to the fact that little or no aqueous effluents are produced, apart from the water necessary for carrying out the reaction,
to provide a manufacturing and/or purification process which may be operated industrially in that it may be carried out under good conditions, in particular in terms of safety of the process.

According to the present invention, at least one stabilizing compound is chosen from the group consisting of phenol derivatives, phenothiazine derivatives, TEMPO derivatives, CB (copper dibutyldithiocarbamate), para-benzoquinone, para-phenylenediamine, diethylhydroxylamine, manganese(II) acetate, sodium nitrite, sodium ascorbate, potassium ascorbate and glutathione.

According to one aspect of the invention, the at least one stabilizing compound is chosen from the group constituted above additionally comprising bis(2,2,6,6-tetramethyl-4-piperidyl) sebacate. Generally, at least one stabilizing compound has a boiling point greater than the boiling point of eugenol; preferably, the boiling point of the stabilizing compound is greater than or equal to Tₑᵤ+1° C., preferably greater than or equal to Tₑᵤ+5° C., more preferentially greater than or equal to Tₑᵤ+10° C. Tₑᵤ represents the boiling point of eugenol at atmospheric pressure.

According to a first specific aspect, the at least one stabilizing compound is a compound of formula (IV) or phenothiazine (PTZ), in particular chosen from the group consisting of catechol, isoeugenol, hydroquinone (HQ), para-methoxyphenol (PMP), tert-butylcatechol (TBC), 2,4-dimethyl-6-(tert-butyl)phenol (Topanol A), 2,4-dinitro-6-(sec-butyl)phenol, 2-methyl-4,6-dinitrophenol, para-nitrosophenol, 2-methyl-4-nitrophenol and phenothiazine (PTZ). According to a second specific aspect of the present invention, at least one stabilizing compound is chosen from the group consisting of hydroquinone (HQ), para-methoxyphenol (PMP), tert-butylcatechol (TBC) and phenothiazine (PTZ). According to a third specific aspect of the present invention, at least one stabilizing compound is chosen from the group consisting of 2,4-dimethyl-6-(tert-butyl)phenol (Topanol A), 2,4-dinitro-6-(sec-butyl)phenol, 2-methyl-4,6-dinitrophenol, para-nitrosophenol and 2-methyl-4-nitrophenol.

According to a fourth specific aspect, at least one stabilizing compound is chosen from the group consisting of vitamin E, butylated hydroxyanisole (BHA), dibutylated hydroxytoluene (BHT), tert-butylhydroquinone (TBHQ), di(tert-butyl)hydroxyanisole (diBHA) and di(tert-butyl)hydroquinone (diTBHQ), in particular chosen from the group consisting of vitamin E, butylated hydroxyanisole (BHA), dibutylated hydroxytoluene (BHT) and tert-butylhydroquinone (TBHQ). According to another specific aspect, the at least one stabilizing compound is chosen from a group according to the first aspect or according to the second aspect or according to the third aspect or a group according to the fourth aspect, said group additionally comprising bis(2,2,6,6-tetramethyl-4-piperidyl) sebacate.

According to one alternative form, at least one stabilizing compound is chosen from the group consisting of vitamin E, butylated hydroxyanisole (BHA), tert-butylcatechol (TBC), bis(2,2,6,6-tetramethyl-4-piperidyl) sebacate, sodium ascorbate, tert-butylhydroquinone (TBHQ), para-methoxyphenol (PMP) and glutathione.

Preferably, the purification process is carried out in the presence of a stabilizer.

According to another preferred aspect, the purification process is carried out in the presence of two stabilizers. Preferably, the two stabilizers are chosen from the group consisting of hydroquinone (HQ), para-methoxyphenol (PMP), tert-butylcatechol (TBC) and phenothiazine (PTZ); very preferably, the process is carried out in the presence of a TBC/PMP, HQ/PMP or TBC/HQ mixture. In particular, the process may be carried out in the presence of a TBC/PMP mixture.

According to another aspect, the present invention relates to a process for the purification of a crude eugenol by distillation in the presence of a stabilizing compound and of an auxiliary compound.

Without however wishing to be committed to any one theory, the inventors have discovered, surprisingly, that the addition of at least one stabilizing compound during the distillation of the crude eugenol makes it possible to shift the temperature zone toward which the thermal decomposition becomes initiated in order to prevent the distillation process from being unstable, indeed even explosive. Thus, the process according to the present invention may be operated under appropriate safety conditions.

According to a specific aspect of the present invention, the crude eugenol is distilled in the presence of at least one auxiliary compound, preferentially chosen from the group consisting of vitamin E, butylated hydroxyanisole (BHA), dibutylated hydroxytoluene (BHT) and tert-butylhydroquinone (TBHQ).

Preferably, the purification process is carried out in the presence of an auxiliary compound chosen from the group consisting of vitamin E, butylated hydroxyanisole (BHA), dibutylated hydroxytoluene (BHT) and tert-butylhydroquinone (TBHQ); preferably, the auxiliary compound is vitamin E.

Preferably, the purification process is carried out in the presence of two auxiliary compounds chosen from the group consisting of vitamin E, butylated hydroxyanisole (BHA), dibutylated hydroxytoluene (BHT) and tert-butylhydroquinone (TBHQ).

According to a specific aspect, the purification process is carried out in the presence of at least one stabilizing compound chosen from the group consisting of phenol derivatives, phenothiazine derivatives, TEMPO derivatives, CB (copper dibutyldithiocarbamate), para-benzoquinone, para-phenylenediamine, diethylhydroxylamine, manganese(II) acetate, sodium nitrite, sodium ascorbate, potassium ascorbate and glutathione and optionally in the presence of at least one auxiliary compound chosen from the group consisting of vitamin E, butylated hydroxyanisole (BHA), dibutylated hydroxytoluene (BHT) and tert-butylhydroquinone (TBHQ). According to a specific embodiment of this aspect, the at least one stabilizing compound is chosen from the above group additionally comprising bis(2,2,6,6-tetramethyl-4-piperidyl) sebacate.

According to the present invention, the total amount of stabilizing compound is greater than or equal to 50 ppm, preferably greater than or equal to 100 ppm, more preferentially greater than or equal to 500 ppm and more preferentially greater than or equal to 1000 ppm, with respect to the amount of eugenol. According to the present invention, the total amount of stabilizing compound is less than or equal to 10% by weight, preferably less than or equal to 5% by weight, more preferentially less than or equal to 1% by weight, more preferentially less than or equal to 5000 ppm, and more preferentially still less than or equal to 2000 ppm, with respect to the amount of eugenol.

According to the present invention, the total amount of auxiliary compound is greater than or equal to 0 ppm, preferably greater than or equal to 100 ppm, more preferentially greater than or equal to 500 ppm and more preferentially greater than or equal to 1000 ppm, with respect to the amount of eugenol. According to the present invention, the total amount of auxiliary compound is less than or equal to 5000 ppm, preferably less than or equal to 2500 ppm, more preferentially less than or equal to 2000 ppm and more preferentially less than or equal to 1000 ppm, with respect to the amount of eugenol.

Advantageously, at least one stabilizing compound may be added at the feeding with eugenol to be purified, preferably may be added to the distillation boiler and/or to the distillation column. The addition may be carried out all at once or in several portions.

According to a specific aspect, the at least one stabilizing compound is added to the distillation boiler. The at least one stabilizing compound is chosen from stabilizing compounds having a boiling point which is greater than that of eugenol; preferably, the boiling point of the stabilizing compound is greater than or equal to Tₑᵤ+1° C., preferably greater than or equal to Tₑᵤ+5° C. and more preferentially greater than or equal to Tₑᵤ+10° C.

According to another specific aspect, the at least one stabilizing compound is added to the distillation column. The at least one stabilizing compound may be chosen from stabilizing compounds having a boiling point which is greater than that of eugenol. Alternatively, the at least one stabilizing compound may be chosen from stabilizing compounds having a boiling point which is less than or equal to that of eugenol; preferably, the boiling point of the stabilizing compound is less than or equal to Tₑᵤ−1° C., preferably less than or equal to Tₑᵤ−5° C. and more preferentially less than or equal to Tₑᵤ−10° C.

Preferably, the distillation is carried out using TBC and PMP. Preferably, the TBC is added to the distillation boiler and the PMP to the distillation column. Alternatively, the TBC and the PMP are added to the distillation boiler.

According to the present invention, the distillation is carried out at a temperature of greater than or equal to 90° C., preferably of greater than or equal to 100° C. and very preferentially of greater than or equal to 110° C., and more preferentially still of greater than or equal to 120° C. According to the present invention, the distillation is carried out at a temperature of less than or equal to 200° C., preferably of less than or equal to 190° C. and very preferentially of less than or equal to 180° C., and more preferentially still of less than or equal to 150° C. According to the present invention, the distillation is carried out at a pressure of greater than or equal to 0.5 mbar, preferably of greater than or equal to 1 mbar, more preferentially of greater than or equal to 5 mbar and more preferentially still of greater than or equal to 10 mbar. According to the present invention, the distillation is carried out at a pressure of less than or equal to 150 mbar, preferably of less than or equal to 100 mbar and very preferentially of less than or equal to 50 mbar.

According to a specific aspect, the distillation also makes it possible to separate the excess of guaiacol, the products of the O-alkylation of eugenol and/or of guaiacol and of ortho-eugenol. The guaiacol used in the preparation of the eugenol may also contain certain impurities, such as veratrole or 6-methylguaiacol; the reaction mixture obtained on conclusion of the allylation reaction may also contain products of allylation of said impurities, in particular allylveratrole or 4-allyl-6-methylguaiacol. The reaction mixture obtained on conclusion of the allylation reaction may also contain products of isomerization of the allyl group, in particular isoeugenol or meta-eugenol (also known under the name of 2-methoxy-5-(prop-2-en-1-yl)phenol).

The present invention also relates to a process which makes possible in particular the separation of eugenol and ortho-eugenol. In particular, the eugenol obtained on conclusion of the distillation is a purified eugenol. In particular, the eugenol obtained on conclusion of the distillation contains an amount of less than or equal to 10% by weight, in particular of less than or equal to 5% by weight, preferentially of less than or equal to 2.5% by weight and more preferentially still of less than or equal to 1% by weight of ortho-eugenol, with respect to the total weight of the composition. According to the present invention, the eugenol obtained on conclusion of the distillation contains an amount of greater than or equal to 0.01% by weight, preferentially of greater than or equal to 0.05% by weight and more preferentially still of greater than or equal to 0.1% by weight of ortho-eugenol, with respect to the total weight of the composition.

The process according to the present invention makes possible in particular the separation of eugenol and guaiacol.

Advantageously, the process according to the present invention makes possible the preparation of eugenol having satisfactory organoleptic properties. In particular, the eugenol obtained according to the process of the invention exhibits similar organoleptic properties to those of the eugenol obtained by extraction of a natural product.

The present invention relates to a process for the preparation of eugenol which comprises a purification process as defined above.

The process for the preparation of eugenol generally comprises a step of preparation of a crude eugenol. This step may in particular be carried out as described in the document FR 2 302 991, in particular by allylation of guaiacol.

The process for the preparation of eugenol comprises a second step in which the crude eugenol is purified by a process of purification by distillation in the presence of at least one stabilizing compound.

The present invention also relates to the use of a process for the purification of crude eugenol as described above for separating eugenol and ortho-eugenol.

In another aspect, the present invention relates to the use of a stabilizing compound for stabilizing a eugenol purification process.

Finally, the present invention relates to a composition comprising eugenol and between 0.1 and 10 000 ppm of at least one stabilizing compound or of at least one stabilizing compound and of at least one auxiliary compound, in which the stabilizing compound is chosen from the group consisting of phenol derivatives, phenothiazine derivatives, TEMPO derivatives, CB (copper dibutyldithiocarbamate), para-benzoquinone, para-phenylenediamine, diethylhydroxylamine, manganese(II) acetate, sodium nitrite, sodium ascorbate, potassium ascorbate and glutathione and the auxiliary compound is chosen from the group consisting of vitamin E, butylated hydroxyanisole (BHA), dibutylated hydroxytoluene (BHT) and tert-butylhydroquinone (TBHQ). In a specific aspect, the above composition additionally comprises bis(2,2,6,6-tetramethyl-4-piperidyl) sebacate.

According to the present invention, the total amount of stabilizing compound in the composition is greater than or equal to 0.1 ppm, preferably greater than or equal to 1 ppm, more preferentially greater than or equal to 10 ppm and more preferentially greater than or equal to 100 ppm, with respect to the amount of eugenol. According to the present invention, the total amount of stabilizing compound is less than or equal to 10 000 ppm, preferably less than or equal to 5000 ppm, more preferentially less than or equal to 2000 ppm, more preferentially less than or equal to 1000 ppm, and more preferentially still less than or equal to 500 ppm, with respect to the amount of eugenol.

According to the present invention, the total amount of auxiliary compound is greater than or equal to 0 ppm, preferably greater than or equal to 0.1 ppm, more preferentially greater than or equal to 1 ppm and more preferentially greater than or equal to 10 ppm, with respect to the amount of eugenol. According to the present invention, the total amount of auxiliary compound is less than or equal to 5000 ppm, preferably less than or equal to 2500 ppm, more preferentially less than or equal to 2000 ppm and more preferentially less than or equal to 1000 ppm, with respect to the amount of eugenol.

According to a specific aspect, at least one stabilizing compound is a compound of formula (IV) or phenothiazine (PTZ) in particular chosen from the group consisting of catechol, isoeugenol, hydroquinone (HQ), para-methoxyphenol (PMP), tert-butylcatechol (TBC), di(tert-butyl)hydroxyanisole (diBHA), di(tert-butyl)hydroquinone (diTBHQ), 2,4-dimethyl-6-(tert-butyl)phenol (Topanol A), 2,4-dinitro-6-(sec-butyl)phenol, 2-methyl-4,6-dinitrophenol, para-nitrosophenol, 2-methyl-4-nitrophenol and phenothiazine (PTZ) and more preferentially is chosen from the group consisting of hydroquinone (HQ), para-methoxyphenol (PMP), tert-butylcatechol (TBC), phenothiazine (PTZ), di(tert-butyl)hydroxyanisole (diBHA) and di(tert-butyl)hydroquinone (diTBHQ). According to one alternative form, at least one stabilizing compound is a compound of formula (IV) or phenothiazine (PTZ) in particular chosen from the group as constituted above additionally comprising bis(2,2,6,6-tetramethyl-4-piperidyl) sebacate.

According to a specific aspect, the composition according to the invention comprises a stabilizing compound.

According to another specific aspect, the composition according to the invention comprises two stabilizing compounds.

According to another specific aspect, the composition according to the invention comprises at least one stabilizing compound chosen from the group consisting of hydroquinone (HQ), para-methoxyphenol (PMP), tert-butylcatechol (TBC) and phenothiazine (PTZ) and at least one auxiliary compound chosen from the group consisting of vitamin E, butylated hydroxyanisole (BHA), dibutylated hydroxytoluene (BHT) and tert-butylhydroquinone (TBHQ).

According to another specific aspect, the composition according to the invention comprises at least one stabilizing compound chosen from the group consisting of 2,4-dimethyl-6-(tert-butyl)phenol (Topanol A), 2,4-dinitro-6-(sec-butyl)phenol, 2-methyl-4,6-dinitrophenol, para-nitrosophenol and 2-methyl-4-nitrophenol and at least one auxiliary compound chosen from the group consisting of vitamin E, butylated hydroxyanisole (BHA), dibutylated hydroxytoluene (BHT) and tert-butylhydroquinone (TBHQ).

According to a specific aspect, the composition according to the present invention comprises at least 90% by weight of eugenol, preferably at least 95% by weight of eugenol, more preferentially at least 99% by weight, more preferentially still at least 99.5% and more preferentially still at least 99.95% by weight of eugenol, with respect to the total weight of the composition.

According to another specific aspect, the content of ortho-eugenol in the composition is less than or equal to 10% by weight, preferably less than or equal to 8% by weight, very preferentially less than or equal to 5% by weight and more preferentially still less than or equal to 1% by weight, with respect to the weight of the composition.

According to a specific aspect, the eugenol/o-eugenol ratio is greater than or equal to 90:10, preferably greater than or equal to 95:5 and very preferentially greater than or equal to 99:1. According to another specific aspect, the content of isoeugenol in the composition is less than or equal to 5% by weight, preferably less than or equal to 2% by weight, very preferentially less than or equal to 1% by weight and more preferentially still less than or equal to 0.5% by weight, with respect to the weight of the composition.

The present invention finally relates to a composition comprising at least 90% by weight of eugenol and up to 10% by weight of ortho-eugenol, with respect to the total weight of the composition. Preferably, the composition comprises at least at least 95% by weight of eugenol, more preferentially at least 99% by weight and more preferentially still at least 99.5% by weight of eugenol, with respect to the total weight of the composition. Preferably, the content of ortho-eugenol in the composition is less than or equal to 10% by weight, preferably less than or equal to 8% by weight, very preferentially less than or equal to 5% by weight and more preferentially still less than or equal to 1% by weight, with respect to the weight of the composition.

Advantageously, the inventors have discovered that the compositions according to the present invention exhibit satisfactory organoleptic properties. In particular, the compositions according to the present invention exhibit organoleptic properties which are similar or at least equivalent to those of the eugenol obtained by extraction of a natural product.

Advantageously, the compositions according to the present invention may be used in perfumery or in aromatic, analgesic, antibacterial or antioxidant compositions. Advantageously, the compositions according to the present invention exhibit properties suitable for their use in perfumery or in aromatic, analgesic, antibacterial or antioxidant compositions, in particular in terms of organoleptic properties.

The examples below are intended to illustrate the invention without, however, limiting it. Should the disclosure of patents, patent applications and publications cited herein by reference conflict with the description of the present patent application to the extent that it risks rendering a term uncertain, the present description shall prevail.

### EXAMPLES

### Examples 1 (Invention) and 2 (Comparative): Differential Scanning Calorimetry Test

A differential scanning calorimetry test is carried out with eugenol in the presence of a stabilizing compound (example 1) or in the absence of any stabilizing compound (comparative—example 2).

TABLE 1

| | Example 1 | | Example 2 | |
|---|---|---|---|---|
| Stabilizer used | TBC | | No stabilizer | |
| Amount | 1000 | ppm | N/A | |
| Beginning of the thermal decomposition | 270° | C. | 110° | C. |
| Amount of energy released | −346 | J/g | −1100 | J/g |

Differential scanning calorimetry carried out on a Mettler Toledo DSC calorimeter; the sample is placed in a gold crucible and heated continuously from 25° C. to 400° C. at the rate of 2° C./min, under a nitrogen flow (50 ml/min).

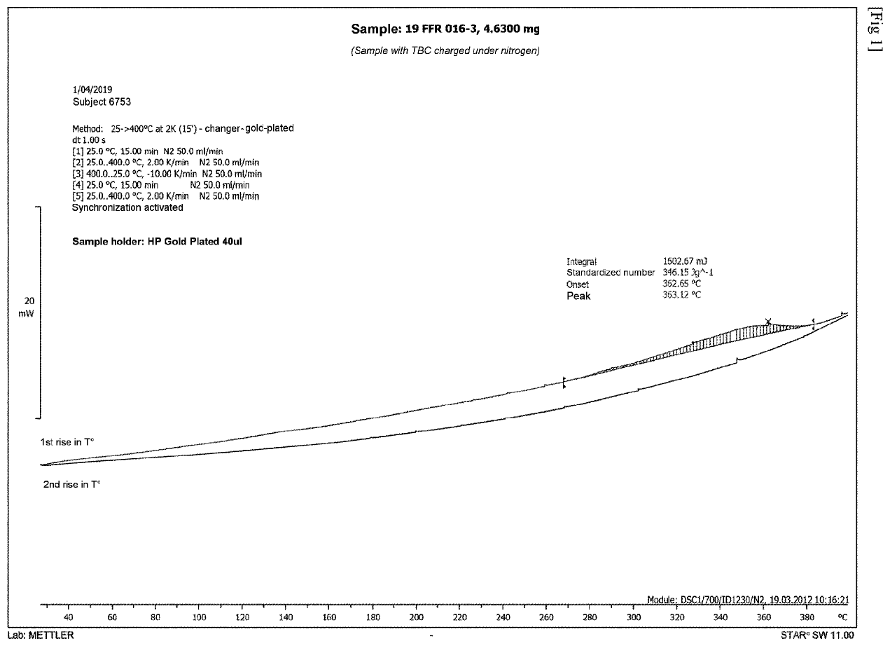

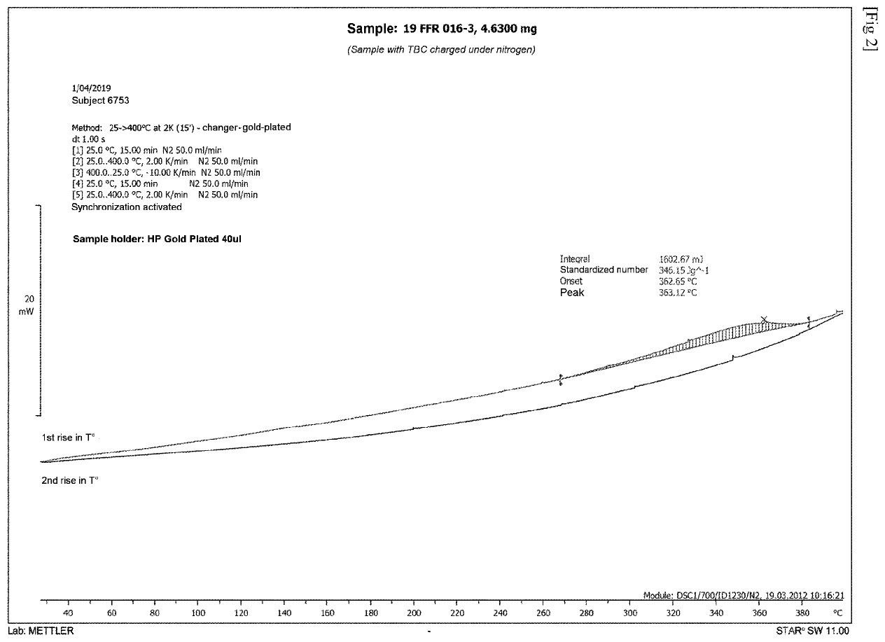

FIG. 2 indicates that, in the absence of stabilizing compound, the thermal decomposition of eugenol begins at 110° C. FIG. 1 indicates that, in the presence of a stabilizing compound, the thermal composition of eugenol is initiated above 270° C., thus making it possible to operate the distillation process under suitable safety conditions.

### Examples 3 (Comparative) and 4 to 11 (Invention): Differential Scanning Calorimetry Tests

A differential scanning calorimetry test is carried out according to examples 1 and 2, with commercial eugenol, originating from a different batch, in the presence of a stabilizing compound (examples 4 to 11) or in the absence of any stabilizing compound (comparative—example 3). The stabilizing compounds tested are: TBC (example 4), vitamin E (example 5), bis(2,2,6,6-tetramethyl-4-piperidyl) sebacate (example 6), sodium ascorbate (example 7), BHA (example 8), TBHQ (example 9), glutathione (example 10) and PMP (example 11).

TABLE 2

| Example | 3 | | 4 | | 5 | | 6 | | 7 | | 8 | | 9 | | 10 | | 11 | |
|---|---|---|---|---|---|---|---|---|---|---|---|---|---|---|---|---|---|---|
| Amount | N/A | | 1000 | ppm | 1000 | ppm | 1000 | ppm | 1000 | ppm | 1000 | ppm | 1000 | ppm | 1000 | ppm | 1000 | ppm |
| Beginning of the | 275° | C. | 295° | C. | 290° | C. | 290° | C. | 285° | C. | 285° | C. | 285° | C. | 280° | C. | 295° | C. |
| thermal | | | | | | | | | | | | | | | | | | |
| decomposition | | | | | | | | | | | | | | | | | | |
| Amount of | −398 | J/g | −170 | J/g | −275 | J/g | −256 | J/g | −235 | J/g | −272 | J/g | −263 | J/g | −290 | J/g | −218 | J/g |
| energy released | | | | | | | | | | | | | | | | | | |

Table 2 indicates that, in the absence of stabilizing compound, the thermal decomposition of eugenol begins at 275° C., releasing 398 J/g. Examples 2 to 9 indicate that, in the presence of a stabilizing compound, the thermal decomposition of eugenol is initiated above 285° C. and releases a significantly lower amount of energy, thus making it possible to operate the distillation process under suitable safety conditions.

1. A process for the purification of eugenol, the process comprising distilling a crude eugenol containing impurities in the presence of at least one stabilizing compound.

2. The process for the purification of eugenol as claimed in claim 1, wherein the stabilizing compound is selected from the group consisting of phenol derivatives, phenothiazine derivatives, TEMPO derivatives, CB (copper dibutyldithiocarbamate), para-benzoquinone, para-phenylenediamine, diethylhydroxylamine, manganese(II) acetate, sodium nitrite, sodium ascorbate, potassium ascorbate, glutathione and bis(2,2,6,6-tetramethyl-4-piperidyl) sebacate.

3. The process for the purification of eugenol as claimed in claim 1, wherein the at least one stabilizing compound has a boiling point greater than the boiling point of eugenol.

4. The process for the purification of eugenol as claimed in claim 1, wherein the total amount of stabilizing compound is between 50 ppm and 10% by weight, with respect to the amount of eugenol.

5. The process for the purification of eugenol as claimed in claim 1, wherein eugenol is separated from ortho-eugenol.

6. The process for the purification of eugenol as claimed in claim 1, wherein the content of ortho-eugenol in the eugenol obtained is less than or equal to 10% by weight, with respect to the total weight of the composition.

7. A process for the separation of eugenol and ortho-eugenol, the process comprising distillation in the presence of at least one stabilizing compound.

8. A process for the manufacture of eugenol, the process comprising the purification process as defined in claim 1.

9. A composition comprising eugenol and between 0.05 and 10 000 ppm of at least one stabilizing compound, wherein the stabilizing compound is selected from the group consisting of phenol derivatives, phenothiazine derivatives, TEMPO derivatives, CB (copper dibutyldithiocarbamate), para-benzoquinone, para-phenylenediamine, diethylhydroxylamine, manganese(II) acetate, sodium nitrite, sodium ascorbate, potassium ascorbate and glutathione; and an auxiliary compound selected from the group consisting of vitamin E, butylated hydroxyanisole (BHA), dibutylated hydroxytoluene (BHT) and tert-butylhydroquinone (TBHQ).

10. A composition comprising eugenol and between 0.05 and 10 000 ppm of at least one stabilizing compound and of an auxiliary compound, wherein the stabilizing compound is selected from the group consisting of phenol derivatives, phenothiazine derivatives, TEMPO derivatives, CB (copper dibutyldithiocarbamate), para-benzoquinone, para-phenylenediamine, diethylhydroxylamine, manganese(II) acetate, sodium nitrite, sodium ascorbate, potassium ascorbate and glutathione; and the auxiliary compound is selected from the group consisting of vitamin E, butylated hydroxyanisole (BHA), dibutylated hydroxytoluene (BHT), tert-butylhydroquinone (TBHQ) and bis(2,2,6,6-tetramethyl-4-piperidyl) sebacate.

11. The composition as claimed in claim 9, wherein the phenol derivative is a compound according to the following formula (IV): 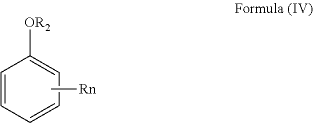
wherein:
R represents a hydroxyl, a linear or branched alkyl, alkenyl, an alkoxy group a nitro group or a nitroso group,
R₂ represents a linear or branched alkyl, and
n is between 0 and 5.

12. The composition as claimed in claim 9, comprising at least 90% by weight of eugenol, with respect to the total weight of the composition.

13. A composition comprising at least 90% by weight of eugenol and up to 10% by weight of ortho-eugenol, with respect to the total weight of the composition.

14. The composition as claimed in claim 10, wherein the phenol derivative is a compound according to the following formula (IV): 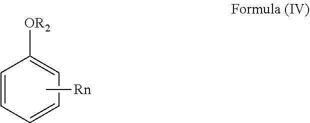
wherein:
R represents a hydroxyl, a linear or branched alkyl, alkenyl, an alkoxy group, a nitro group or a nitroso group,
R₂ represents a linear or branched alkyl, and
n is between 0 and 5.

15. The composition as claimed in claim 10, comprising at least 90% by weight of eugenol, with respect to the total weight of the composition.
